(19) United States
(12) Patent Application Publication
(10) Pub. No.: US 2007/0036844 A1
(43) Pub. Date: Feb. 15, 2007

(54) Porous materials having multi-size geometries

(76) Inventors: Peter X. Ma, Ann Arbor, MI (US); Victor J. Chen, Ann Arbor, MI (US)

Correspondence Address: JULIA CHURCH DIERKER;DIERKER & ASSOCIATES, P.C., 3331 W. BIG BEAVER RD. SUITE 109, TROY, MI, 48084-2813, US

(21) Appl. No.: 11/496,238

(22) Filed: Jul. 31, 2006

Related U.S. Application Data
(60) Provisional application No. 60/704,477, filed on Aug. 1, 2005.

(51) Int. Cl. A61K 9/70; B29C 44/04

(52) U.S. Cl. 424/443; 264/45.1

(57) ABSTRACT

A method for forming a porous three-dimensional (3-D) object includes creating a mold from a negative replica of the 3-D object, the 3-D object having a first size and at least one predetermined feature, and then casting a flowable material into and/or onto the mold. The method further includes forming pores of a second size and/or a third size in the flowable material, thereby forming the porous 3-D object.

### CROSS REFERENCE TO RELATED APPLICATIONS

This application claims the benefit of U.S. Provisional Patent Application Ser. No. 60/704,477 filed on Aug. 1, 2005.

### STATEMENT REGARDING FEDERALLY SPONSORED RESEARCH OR DEVELOPMENT

This invention was made in the course of research supported by a grant from the National Institutes of Health (NIH) and the National Institute of Dental and Craniofacial Research (NIDCR), Grant Nos. DEO15384 and DEO14755; and from the National Institutes of Health (NIH), Grant No. T32HD07505. The U.S. government has certain rights in the invention.

### BACKGROUND

The present disclosure relates generally to porous materials, and more particularly to porous materials having multi-size geometries.

Tissue engineering aims to solve the problems of organ and donor shortages. One approach is to use a three-dimensional (3-D) biodegradable scaffold to seed cells, which will promote tissue formation. To emulate the fibrous morphology in type I collagen, materials have been electrospun or self-assembled to form scaffolds. Some of the challenges with these techniques are that electrospinning typically forms two-dimensional sheets (thus limiting the ability to create 3-D scaffolds); and self-assembling materials usually form hydrogels, generally limiting the geometric complexity of the scaffold.

Porous materials may be made using many different fabrication technologies, some examples of which include sintering, stretching, extrusion, self-assembly, phase inversion, phase separation, porogen-leaching, gas-foaming, etching, and solid free-form fabrication techniques. In general, each fabrication technology may generate certain pore sizes and shapes, and they are often not mutually compatible.

As such, it would be desirable to provide a 3-D porous material with multi-size geometries.

### SUMMARY

A method for forming a porous three-dimensional (3-D) object includes creating a mold from a negative replica of the 3-D object, the 3-D object having a first size and at least one predetermined feature, and then casting a flowable material into and/or onto the mold. The method further includes forming pores of a second size and/or a third size in the flowable material, thereby forming the porous 3-D object.

### BRIEF DESCRIPTION OF THE DRAWINGS

Objects, features and advantages of embodiments of the present disclosure will become apparent by reference to the following detailed description and drawings, in which:

FIG. 1A is a semi-schematic perspective view of an embodiment of a negative mold design used for scaffold casting; solid struts in the mold eventually become the open pores in the final scaffold;

FIG. 1B is a SEM micrograph of a 3-D poly(L-lactic acid) (PLLA) scaffold created from reverse solid-freeform fabrication showing an overview of a nano-fibrous (NF) scaffold with micro-pore structure in the struts, scale bar 500 μm;

FIG. 1C is a SEM micrograph of a 3-D PLLA scaffold created from reverse solid-freeform fabrication showing the fibrous morphology of the NF scaffold pore walls, scale bar 2 μm;

FIG. 1D is a SEM micrograph of a 3-D PLLA scaffold created from reverse solid-freeform fabrication showing the overview of a solid-walled (SW) scaffold showing micro-pore structure in the struts, scale bar 500 μm;

FIG. 1E is a SEM micrograph of a 3-D PLLA scaffold created from reverse solid-freeform fabrication showing the solid nature of the SW scaffold pore walls, scale bar 2 μm;

FIG. 2A is a semi-schematic diagram of a human ear reconstruction image from histological sections;

FIG. 2B is an NF scaffold formed using the image of FIG. 2A, scale bar 10 mm;

FIG. 2C is a semi-schematic diagram of a human mandible reconstruction image obtained from CT-scans, the enlarged segment showing the reverse image of the bone fragment to be engineered;

FIG. 2D is an NF scaffold formed using the image of FIG. 2C, scale bar 10 mm;

FIG. 2E is a SEM micrograph of interconnected spherical pores within the mandible segment of FIG. 2D, scale bar 500 μm;

FIG. 2F is a SEM micrograph of the NF pore morphology of a spherical pore of FIG. 2E, scale bar 5 μm;

FIG. 3A is a histological section of an overview of a Hematoxylin and Eosin-Phloxine (H&E) stained NF scaffold after seeding with MC3T3-E1 osteoblasts and cultured under differentiation conditions for about 6 weeks, scale bar 500 μm;

FIG. 3B is a histological section of an overview of an H&E stained SW scaffold after seeding with MC3T3-E1 osteoblasts and cultured under differentiation conditions for about 6 weeks, scale bar 500 μm;

FIG. 3C is a histological section of a center region of an H&E stained NF scaffold after seeding with MC3T3-E1 osteoblasts and cultured under differentiation conditions for about 6 weeks, scale bar 100 μm;

FIG. 3D is a histological section of a center region of an H&E stained SW scaffold after seeding with MC3T3-E1 osteoblasts and cultured under differentiation conditions for about 6 weeks, scale bar 100 μm;

FIG. 3E is a histological section of a center region of a Von Kossa's silver nitrate stained NF scaffold after seeding with MC3T3-E1 osteoblasts and cultured under differentiation conditions for about 6 weeks, scale bar 500 μm, * denotes a PLLA scaffold, # denotes a scaffold pore, an arrow denotes mineralization;

FIG. 3F is a histological section of a center region of a Von Kossa's silver nitrate stained SW scaffold after seeding with MC3T3-E1 osteoblasts and cultured under differentiation conditions for about 6 weeks, scale bar 500 μm, * denotes a PLLA scaffold, # denotes a scaffold pore, an arrow denotes mineralization;

FIG. 4A is a diagram depicting osteocalcin (OCN) expression in NF and SW scaffolds after 2 and 6 weeks of culture under differentiation conditions;

FIG. 4B is a diagram depicting bone sialoprotein (BSP) expression in NF and SW scaffolds after 2 and 6 weeks of culture under differentiation conditions;

FIG. 4C is a diagram depicting Type I collagen (COL) expression in NF and SW scaffolds after 2 and 6 weeks of culture under differentiation conditions; and

FIG. 5 is a diagram depicting the short-term in vitro osteoblastic proliferation behavior on NF and SW scaffolds after seeding with one-half million MC3T3-E1 cells.

### DETAILED DESCRIPTION OF THE EMBODIMENTS

A series of compositions and methods to form porous materials with complex geometries on multiple size scales have been unexpectedly and fortuitously discovered. Current manufacturing methods, for example computer assisted manufacture (CAM) methods, form an object directly from the material used in the manufacturing process, and the structure of that formed object generally cannot be modified. In sharp contrast, it has been advantageously found, and disclosed herein, that by instead using a material from the CAM process to create a negative replica/reverse image mold of the desired 3-D structure, the casting materials (and/or the mold prior to introduction of the casting materials) may be manipulated to form a 3-D object having a predetermined porous structure with random and/or predesigned pores. Further, if desired, the porous 3-D object may be formed with predetermined properties advantageous for a particular application/end use.

This disclosure describes new technologies for the design and fabrication of complex structures/3-D objects with various geometries on multiple size scales. As described further hereinbelow, the materials forming the 3-D objects may be of any suitable type, including but not limited to at least one of synthetic polymers, natural macromolecules/polymers, organic compounds, inorganic compounds, metals, and combinations thereof, as long as the materials may flow and be cast in/on a mold under predetermined conditions.

A method for forming a porous three-dimensional (3-D) object according to one embodiment includes creating a mold from a negative replica of the 3-D object, the 3-D object having a first size and at least one predetermined feature (e.g., nano-fibrous, nano-pores, micro pores, macropores, nano-patterns, micro-patterns, or the like, or combinations thereof). It is to be understood that the creation of the negative replica mold may be by any suitable methods, including but not limited to CAM, impression and casting, manual building, machine milling, or the like, or combinations thereof. One example of a suitable CAM method is solid free form (SFF) fabrication.

The method further includes casting/introducing a flowable/casting material into and/or onto the mold. Predetermined pores/porous structures of a second size and/or a third size are formed in the flowable material, thereby forming the porous 3-D object. It is to be understood that the method(s) for forming the predetermined pores may be any suitable methods and/or combinations of methods, some examples of which are recited hereinbelow. It is to be further understood that the predetermined pores/porous structures may be random, uniform, predesigned, and/or combinations thereof. One non-limitative example of predesigned pores includes predesigned interconnected, open pores.

It is to be understood that the pore forming step may be completed before the flowable material is introduced into/onto the mold (such as, for example, by forming air bubbles in liquid flowable materials, forming gaps between particles in powder flowable materials, and/or the like), and/or after the flowable material is introduced into/onto the mold. If the pore forming is done before introduction into the mold, the pores may in some instances be somewhat less controlled, but it is believed that this would still be advantageous over current methods.

In an embodiment, at least one of the first size, the second size and the third size are different from at least another of the first size, the second size and the third size. In an alternate embodiment, the first size is greater than the second size, and the second size is greater than the third size. It is to be understood that the first, second and third sizes depend, at least in part, on the desirable structure of the 3-D object to be formed, the flowable material used, or the like, or combinations thereof. As a non-limiting example, the 3-D object is a macro object having a first size greater than or equal to 10⁻³ m, and the flowable material is treated to have pores of second and third sizes (e.g., micro pores ranging from about 10⁻⁶ m to about 10⁻³ m and nano pores ranging from about 10⁻⁹ m to about 10⁻⁶ m).

The method may further include removing the mold from the porous 3-D object by any suitable method(s). Non-limiting examples of such removal techniques include dissolution, melting, sublimation, evaporation, burning, and/or by any other suitable means, and/or combinations thereof. It is to be understood that the removal technique selected may be based on, at least in part, the material used to form the 3-D object.

Still further, before creating the mold, the method may also include designing and/or obtaining a 3-D image of the 3-D object. It is to be understood that this design and/or obtaining may be accomplished by any suitable method(s), including but not limited to computer assisted design (CAD), computed tomography (CT) scanning, and/or the like, and/or combinations thereof.

In a further embodiment, the 3-D object has at least one of predetermined mechanical properties (e.g., a tissue engineering scaffold that is able to maintain structural integrity under cell culture or implantation conditions, 3D implants that have the mechanical properties of body parts, etc.); predetermined physical properties (e.g., hydrophilicity, melting point, glass transition temperature, crystallinity, porosity, surface area, etc.); predetermined physiological properties (e.g., artificial kidney filtration function, heart valve prosthesis that allows directional fluid flow, etc.); predetermined biological properties (e.g., biocompatibility; allowing cell adhesion, proliferation, and/or differentiation; facilitating tissue formation; allowing or enhancing adsorption of protein or biological agents; preventing adhesion of certain cells, proteins or biological molecules; preventing bacteria adhesion; etc.); predetermined chemical properties (e.g., functional groups that can react with other molecules or agents, etc.); and/or combinations thereof.

Some further non-limitative embodiments will now be described. A computer is used to design (CAD) a macro object (size: greater than or equal to 10⁻³ m) that contains certain features. A reverse image (negative replica) of this object is then fabricated using a CAM technique (e.g. solid free-form fabrication) to form a mold. A casting/flowable material is poured into/onto this mold. The material is then treated to form pores of a predetermined size (micro pores: between about 10⁻⁶ m and about 10⁻³ m; and/or nano pores: between about 10⁻⁹ m and about 10⁻⁶ m). It is to be understood that the pores may be formed by any suitable means, non-limitative examples of which include phase separation (solid-liquid and/or liquid-liquid), evaporation, sublimation, etching, gas generation, particulate-leaching, and/or the like, and/or combinations thereof. If more than one pore-forming method/treatment is used, it is to be understood that the treatments may be performed simultaneously or in sequence. Afterwards, the mold may be removed by dissolution, melting, sublimation, and/or by any other suitable means, and/or combinations thereof. In this manner, a 3-D object of the desired macro shape is formed with micro- and/or nano-pores.

In an alternate embodiment, a porogen material, each unit of which has a predetermined geometry (examples of which include any regular and/or non-regular geometric shape, e.g. spheres, with each unit having substantially the same or a different shape than an other unit) is introduced to the mold (randomly and/or in a predesigned configuration) before the flowable material is introduced thereto. The flowable material is then poured into/onto this mold containing the porogen material. The flowable material is then treated to form the pores of the predetermined size(s) as discussed above. Afterwards, the mold and the added porogen material may be removed by dissolution, melting, sublimation, and/or by any other suitable means, and/or combinations thereof. In this manner, a 3-D object of the desired macro shape is formed with predesigned pores from the porogen, plus the micro- and/or nano-pores.

In yet a further alternate embodiment, the mold plus porogen material assembly is treated by physical and/or chemical means to form connections between the added porogen units and/or between the porogen and the mold. Non-limiting examples of physical means to form the connections include mechanical compression, thermal melting, or the like, or combinations thereof. Non-limiting examples of chemical means to form the connections include partially dissolving, partially reacting, etching, or the like, or combinations thereof. The flowable material is then poured into/onto this porogen/mold assembly. The flowable material is then treated as discussed above to form the pores of the predetermined size(s) as discussed above. Afterwards, the mold and the added porogen material may be removed by one or more of any suitable method, including but not limited to the methods discussed herein. In this manner, a 3-D object of the desired macro shape is formed with predesigned inter-connected, open pores from the porogen, plus the micro- and/or nano-pores.

Further, in an alternate embodiment, instead of designing the image, the image is obtained from an existing object using any suitable methods. One example of such a suitable method is a computed-tomography (CT) scan. The existing object may be any suitable object, including but not limited to a human organ, machine part, a series of histological slides of a tissue, etc. A reverse image (negative replica) of this existing object is then fabricated using, for example, a computer-assisted manufacture (CAM) technique to form a mold. Then any of the methods/treatments as discussed herein may be used to form the 3-D object.

In still a further embodiment, an image of an existing object is obtained as discussed above. In addition, pores with designed shapes are incorporated into the image. In an embodiment, graphic software and computers are used to incorporate pores into the image. A reverse image (negative replica) of this modified image of an existing object is then fabricated, and the method(s) may proceed as discussed herein. In this manner, a 3-D object with the shape of an existing object is formed with computer-designed pore shapes plus porogen-defined pore shapes and/or porogen-defined inter-connected open pores and/or micro- and/or nano-pores.

It is to be understood that the fabricated porous materials as disclosed herein may be used in any of a variety of applications, including but not limited to biomedical applications, industrial applications, household applications, and/or the like, and/or combinations thereof. In the biomedical field, these porous materials may be used as scaffolds for tissue engineering, wound dressings, drug release matrices, membranes for separations and filtration, artificial kidneys, absorbents, hemostatic, and/or the like. In industrial and household applications, these porous materials may be used as insulating materials, packaging materials, impact absorbers, liquid or gas absorbents, membranes, filters, and/or the like.

As mentioned briefly hereinabove, it is to be understood that the casting/flowable material(s) may include any suitable material(s) for flowing and casting into/onto a mold under predetermined conditions. Examples of such materials include, but are not limited to at least one of synthetic polymers, natural macromolecules/polymers, organic compounds, inorganic compounds, metals, and combinations thereof. Further suitable examples of materials are listed immediately below.

Some exemplary polymers suitable for the present disclosure include at least one of natural or synthetic hydrophilic polymers, natural or synthetic hydrophobic polymers, natural or synthetic amphiphilic polymers, degradable polymers, partially degradable polymers, non-degradable polymers, and combinations thereof.

Some exemplary, non-limitative non-degradable water soluble (hydrophilic) polymers include polyvinyl alcohol, polyethylene oxide, polymethacrylic acid (PMAA), polyacrylic acid, polyethylene glycol, alginate, collagen, gelatin, hyaluronic acid, and mixtures thereof. It is to be understood that the natural macromolecules such as alginate, collagen, gelatin and hyaluronic acid are generally not degradable unless treated with appropriate enzymes.

Some exemplary, non-limitative non-degradable water insoluble (hydrophobic) polymers include polytetrafluoroethylene (PTFE), polyvinylchloride (PVC), polyamides (PA, Nylons), polyethylenes (PE), polysulfones, polyethersulfone, polypropylenes (PP), silicon rubbers, polystyrenes, polycarbonates, polyesters, polyacrylonitrile (PAN), polyimides, polyetheretherketone (PEEK), polymethylmethacrylate (PMMA), polyvinylacetate (PVAc), polyphenylene oxide, cellulose and its derivatives, polypropylene oxide (PPO), polyvinylidene fluoride (PVDF), polybutylene, and mixtures thereof.

Some exemplary, non-limitative degradable polymers include at least one of poly(lactide-co-glycolide) (PLGA), poly(lactide) (PLA), poly(L-lactic acid) (PLLA), poly(D,L-lactic acid) (PDLLA), polyglycolic acid (PGA), polyanhydrides, poly(ortho ethers), and mixtures thereof.

Further exemplary, non-limitative degradable polymers (which may or may not be water soluble) include polyamino acids, engineered artificial proteins, natural proteins, biopolymers, and mixtures thereof.

Partially degradable polymers may be formed by any suitable means, one example of which is through the block copolymerization of a degradable polymer with a non-degradable polymer. Examples of non-degradable polymers are disclosed hereinabove. A few non-limitative examples of partially degradable polymers include a block copolymer of PMMA/PLA; and a block copolymer of polyethylene oxide/PLA.

As discussed above, the polymers may be synthetic or natural. They may be homopolymers (with one structural unit) or copolymers (with two or more structural units). The copolymers may be random copolymers, block copolymers, graft copolymers, and/or mixtures thereof. They may be one single polymer type or polymer blends. Further, the materials may also be a composite of polymeric and non-polymeric materials. Yet still further, it is to be understood that chemically or biologically active and/or inert materials may be included as additives or as major components. These polymers may be physically, chemically, and/or biologically modified to improve certain properties or function. It is to be yet further understood that such modification may be carried out before fabrication (raw materials) or after fabrication of the porous materials.

It is to be understood that any suitable solvents may be used in embodiments of the present disclosure pertaining to non-degradable porous materials, providing the solvent(s) performs suitably within the context of embodiment(s) of the present method. In an embodiment of the present disclosure, the solvent includes at least one of water, acetic acid, formic acid, tetrahydrofuran (THF), dimethylsulfoxide (DMSO), dioxane, benzene, and/or the like, and/or mixtures thereof.

Further, it is to be understood that any suitable solvents may be used in embodiments of the present disclosure pertaining to degradable or partially degradable porous materials, provided that the solvent(s) performs suitably within the context of embodiment(s) of the present method. In an embodiment, a mixed solvent is used at a ratio of higher than about 1:1, first solvent to second solvent. In a further embodiment, the first solvent includes dioxane, benzene, and mixtures thereof; and the second solvent includes pyridine, tetrahydrofuran (THF), and mixtures thereof. It is to be understood that dioxane may be mixed with pyridine and/or THF; and that benzene may be mixed with pyridine and/or THF. In an alternate embodiment, the ratio of first solvent to second solvent is about 2:1; and in a further alternate embodiment, the ratio of first solvent to second solvent is about 3:1.

It is contemplated as being within the purview of the present disclosure to use any suitable flowable organic materials, as long as they are capable of forming a solid. Some non-limitative examples of such organic compounds include at least one of naphthalene, fructose, glucose, and/or the like, and/or combinations thereof.

Yet further, it is to be understood that any inorganic material(s) which are suitable for casting and solidification (such as through sintering, for example) and/or are suitable for forming a composite material with one or more of the polymeric materials listed above (one non-limitative example of which is an ionomer composite material) are contemplated as being within the purview of the present disclosure. Some non-limitative examples of such materials include at least one of hydroxyapatite (HAP), carbonated hydroxyapatite, fluorinated hydroxyapatite, various calcium phosphates (CAP), bioglass, other glass materials (one example of which is glass powder (GP)), salts, oxides, silicates, and/or the like, and/or mixtures thereof.

Any metal material(s) suitable for casting and solidification (such as through sintering, for example, with powder materials) and/or for forming a composite material with one or more of the polymeric materials listed above are also contemplated as being within the purview of the present disclosure. Some exemplary metal materials include, but are not limited to powders and/or melts of gold, silver, platinum, palladium, titanium, nickel, cobalt, chromium, iron, copper, aluminum, indium, tin, lead, beryllium, zinc, silicon, gallium, mercury, molybdenum, magnesium, manganese, vanadium, alloys thereof, and/or combinations thereof.

If the inorganic materials and/or metal materials are used in composites, the solvents are generally for dissolving the polymers. Some suitable examples of solvents include, but are not limited to tetrahydrofuran (THF), chloroform, dioxane, any other suitable solvents recited herein, and/or the like, and/or mixtures thereof.

A recently developed phase-separation technique generates porous polymeric materials (porosity is typically higher than 80 or 90%) with a unique nano fibrous structure (an average fiber diameter ranging from 50 nm to 500 nm). The structure includes nano-pores and/or micro-pores. See Ma, P. X. and R. Zhang, Fibrillar Matrices, in U.S. Pat. No. 6,146,892, which patent is incorporated by reference herein in its entirety.

With the recently developed techniques, dissolution/gelation (phase-separation)/solvent exchange (may be optional)/freezing/freeze-drying are some illustrative sequences to create porous nano fibrous structure(s). The structure(s) includes nano-pores and/or micro-pores.

The structures and properties of the porous materials generally depend at least on either the polymer/solvent systems and/or the phase-separation conditions; such as type of polymer(s), type of solvent(s), mixture ratio of two or more types of polymer(s) and/or solvent(s), polymer concentration, phase-separation temperature, etc.

To further illustrate embodiment(s) of the present disclosure, various examples are given herein. It is to be understood that these examples are provided for illustrative purposes and are not to be construed as limiting the scope of the disclosed embodiment(s).

### EXAMPLES

These examples disclose a technique that combines phase separation of poly(L-lactic acid) (PLLA) solutions with reverse solid free-form (SFF) fabrication to form 3-D nano-fibrous (NF) scaffolds with complex geometries. This approach allows for the fabrication of NF matrices while having substantially precise control of internal pore size and structure, as well as external scaffold shape including architectures generated from CAD and/or computed-tomography (CT) scans and histological sections. In vitro cell cultivation experiments show improved proliferation, differentiation, and mineralization on NF scaffolds.

The 3-D configuration and nanometer-scaled morphology in the extracellular matrix (ECM) have been suggested to affect cell behavior in several tissues. While type I collagen has been used as a scaffolding material in tissue regeneration, there may be a significant lack of control regarding its mechanical properties, degradation rate, and batch-to-batch consistency, as well as the potential for pathogen transmission. Much effort has been put into creating scaffolds with nanometer-scaled fibers out of synthetic polymers, but to date, there has been little success in creating 3-D NF matrices with complex, reproducible architecture. This disclosure and these experiments demonstrate the ability to fabricate 3-D NF matrices (non-limitative examples of which include PLLA matrices) with well-defined pore structures created from a reverse SFF technique, and the bone tissue-forming capabilities of these scaffolds. By combining SFF with polymer phase separation, the present disclosure shows the capability to design and create highly reproducible scaffolds with intricate architectures on three different orders of magnitude: macro (millimeter-sized external shapes), micro (micrometer-sized internal pores), and nano (nanometer-sized fibers) size scales.

A negative mold may be created from SFF, into which a PLLA solution can be poured and thermally phase separated to create the NF structures. The mold may subsequently be dissolved to leave the 3-D fibrous scaffold. Without being bound to any theory, it is believed that the NF morphology in the scaffolds would mimic the morphological functions of type I collagen, thus providing a favorable environment for bone tissue formation.

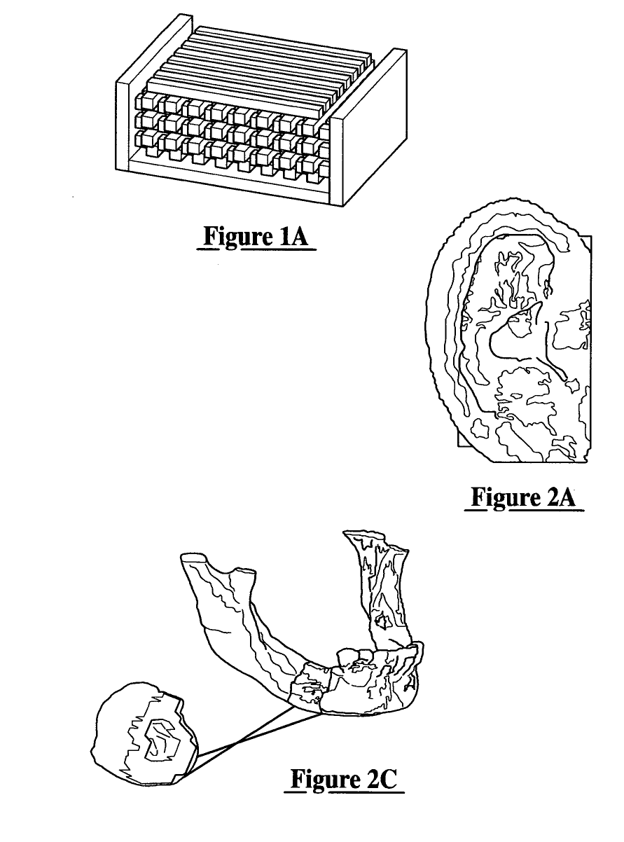

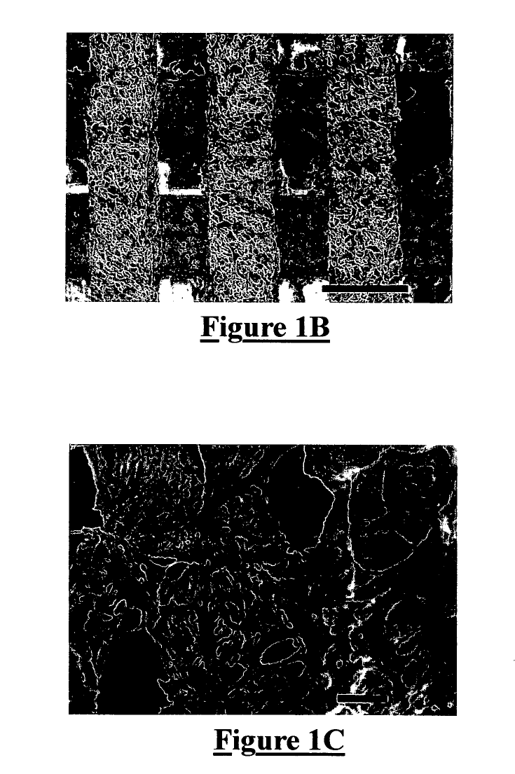

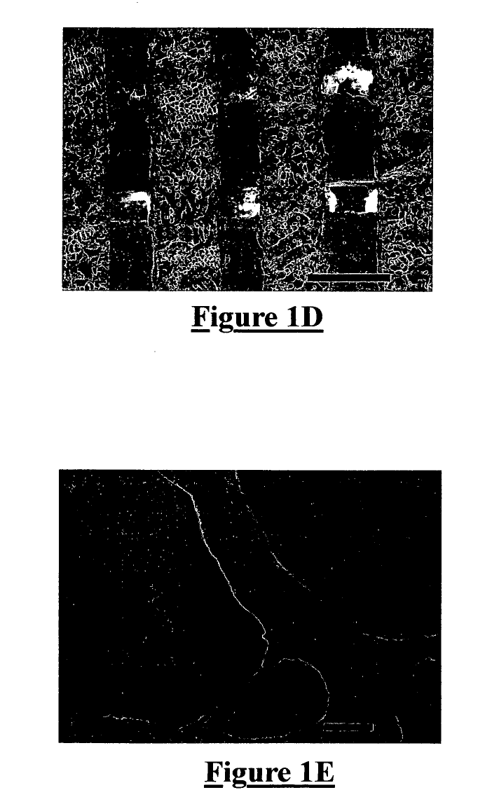

For scaffolds used in cell studies, the external shape had the dimensions (L×W×H) 6.6×6.6×2.45 mm, designed to fit into specially-designed Teflon® cell seeding wells. Internal designs for cell culture scaffolds consisted of partially-overlapped orthogonally stacked layers of parallel rectangular channels. Molds were printed on a 3-D printer, and were designed to have open channels of (W×H) 400×300 μm and closed struts of (W×H) 350×300 μm (a semi-schematic example of which is shown in FIG. 1A). Dimensions for the channel spacing within the scaffold were designed to allow for proper diffusion of nutrients or waste products in or out through the porous scaffold struts, as well as potential angiogenesis when implanted in vivo.

For NF scaffolds, a 9% (wt/v) solution of PLLA in 4:1 (v/v) dioxane:methanol was used. The polymer solution was cast into the mold, and polymer/mold composite was phase separated at −20° C. Solvent exchange, mold leaching, and freeze drying completed the process of scaffold fabrication (more details described below under the heading “Methods”). Upon phase separation, micron-sized pores are formed within the struts due to dioxane crystallization. Scanning electron microscopy (SEM) images show the micron-sized pores within the larger struts and the NF pore wall morphology, respectively (FIGS. 1B and 1C). As a control, solid-walled (SW) scaffolds with similar pore structures were fabricated using a 9% (wt/v) PLLA/dioxane solution. SEM images show the similarities in the micro-pore structure between the NF and SW scaffolds, along with the smooth nature of the pore walls (FIGS. 1D and 1E).

To show the differences in the total amount of available surface area between the two scaffold types, the BET surface area was measured by N₂ adsorption experiments at liquid nitrogen temperature. The specific surface area of the NF scaffolds was about 107.4 m²/g, significantly higher than the 8.2 m²/g seen in the SW scaffolds. In a previous study, it was shown that the increased surface area substantially promoted more protein adsorption and initial cell attachment.

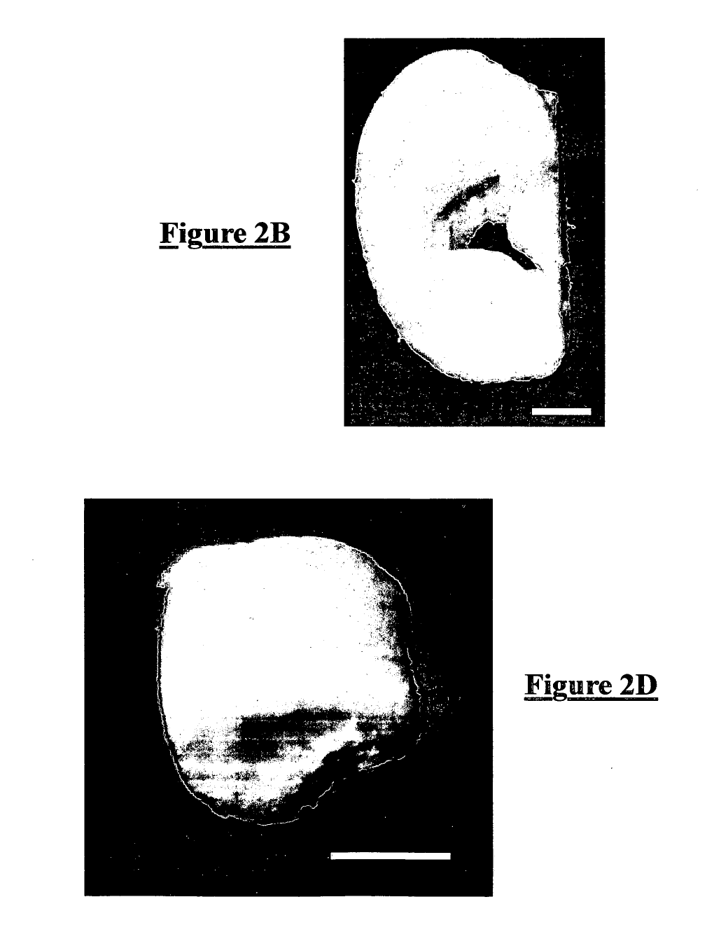

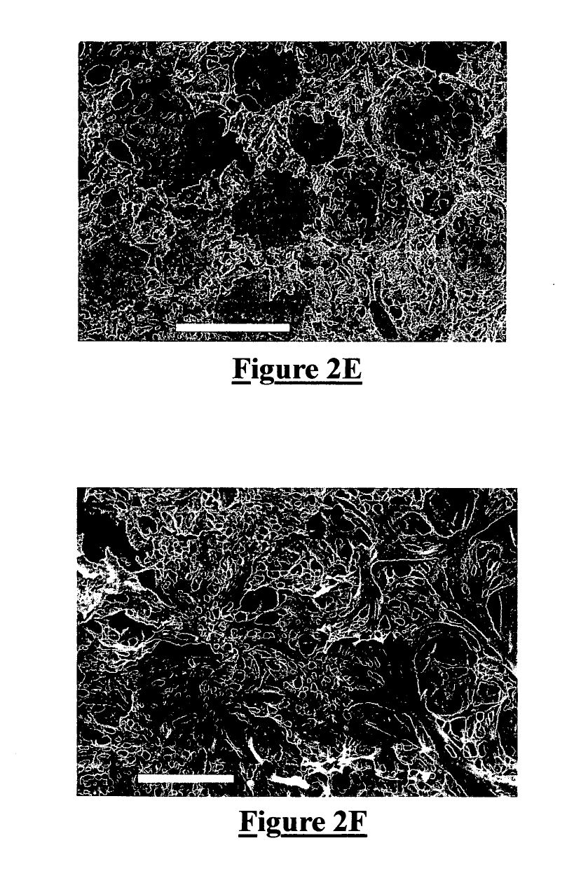

To demonstrate the versatility of the fabrication technique, NF scaffolds were also created using CT scans or histological sections of human anatomical parts. Three-dimensional reconstructions of the scans were computer-generated and converted into stereo lithography (STL) data before proceeding with the SFF process. STL models and the NF PLLA scaffolds created from the molds of a human ear and a human mandible segment are shown (FIGS. 2A-2D). The wax molds were packed with paraffin spheres and heat-treated prior to casting the polymer solution to provide an interconnected spherical pore network with NF pore walls (FIGS. 2E and 2F).

Next, the abilities were compared of the NF and SW scaffolds to support the in vitro growth of bone tissue, and the cellular response to the scaffold surfaces was examined. 2×10⁶ MC3T3-E1 mouse osteoblasts were seeded onto both scaffolds, and the samples were cultured for varying times and conditions depending on the experiment. In short-term studies, it was found that the osteoblasts proliferated more rapidly on the NF scaffolds compared with the SW scaffolds.

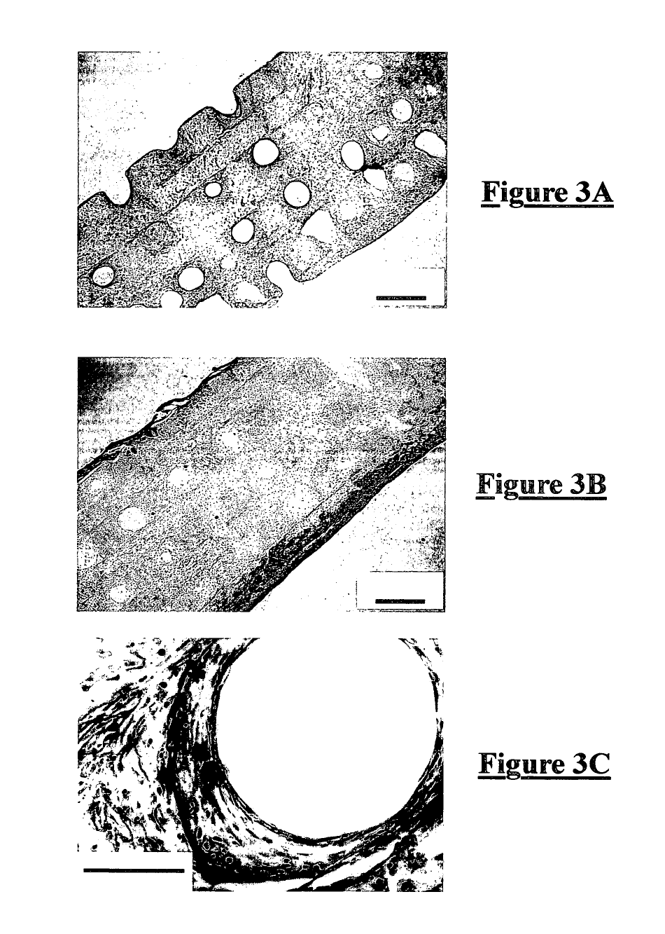

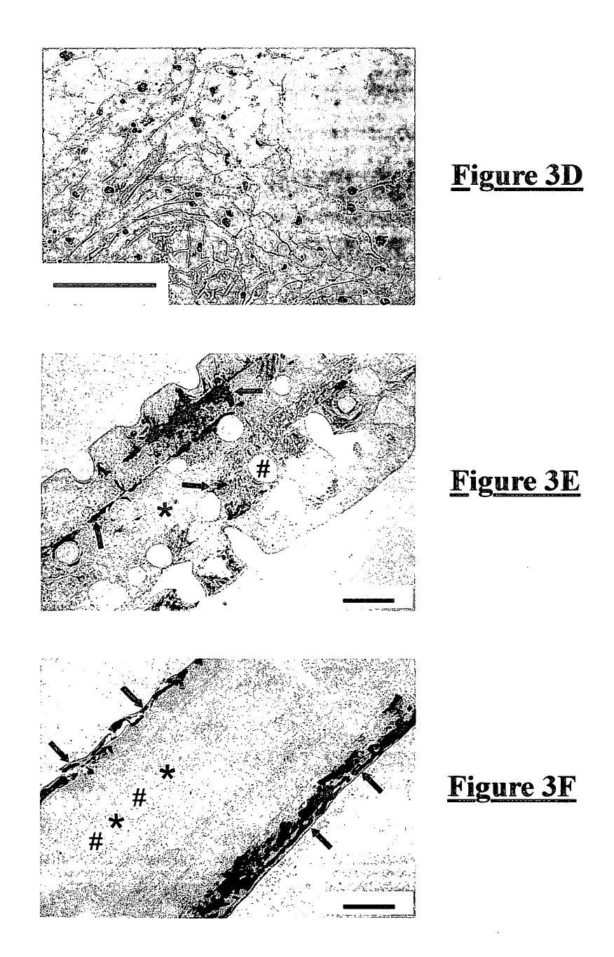

In differentiation studies, the samples were cultured in alpha minimum essential media supplemented with ascorbic acid and β-glycerol phosphate for time periods up to 6 weeks. Histological sections of the samples show after 6 weeks of culture that NF scaffolds produced more organized cellular tissues with increased ECM throughout the scaffold than the SW scaffolds (FIG. 3). This was especially evident near the center of the scaffolding (FIGS. 3C and 3D) where the osteoblasts continued to remain viable even as the pore openings in the scaffold were filled with ECM. Since the fibrous mesh in the NF pore walls allows for the diffusion of nutrients and waste products into and out of the scaffold, the new tissue produced within these scaffolds is able to remain healthy. Although the SW pore walls had micron-sized pores from the dioxane crystallization, the absence of nano-fibers did not allow for diffusion through the SW scaffold struts, and the only tissue that is able to survive is on the outer surfaces.

The NF scaffolds also produced mineralization throughout the scaffold, whereas the SW scaffolds tended to mineralize near the surface as is generally typical of tissue engineering constructs (FIGS. 3E and 3F). Compared with SW scaffolds, quantification of the overall mineral content showed that NF scaffolds increased mineralization by about >80% (mineral content was 3.3±0.3 μmol per NF scaffold vs. about 1.8±0.4 μmol per SW scaffold), and qualitatively comparing the center regions of the scaffolds, NF samples showed a considerably higher content of mineralization.

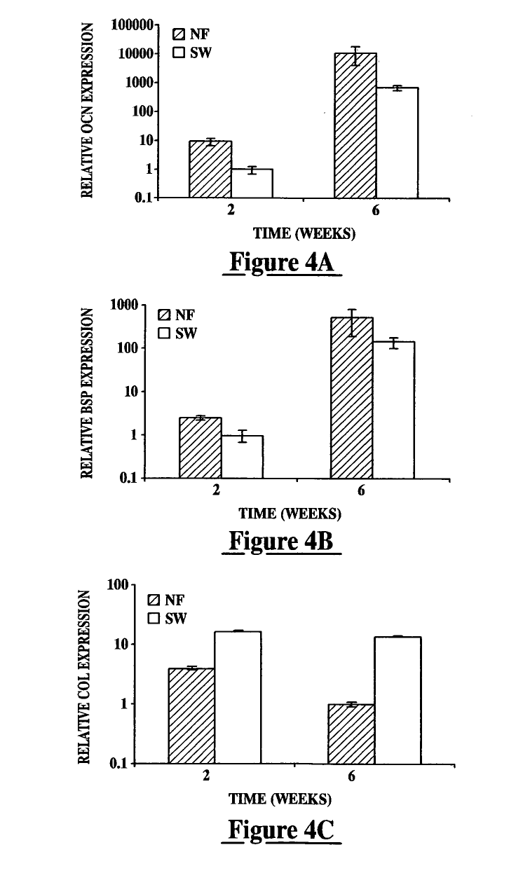

The expression of mRNAs encoding osteocalcin (OCN), bone sialoprotein (BSP), and type I collagen (COL), all markers for bone differentiation, in the scaffolds after 2 and 6 weeks of culture under differentiation conditions (FIGS. 4A-4C) were examined. After 2 weeks, OCN and BSP levels were significantly increased in NF scaffolds compared with SW scaffolds. While OCN and BSP levels increased in both types of scaffolds after 6 weeks, mRNA contents in the NF scaffolds continued to be expressed at significantly higher levels compared with SW scaffolds. When examining levels of type I collagen in the scaffolds, COL expression decreased in both types of scaffolds from 2 weeks to 6 weeks as a consequence of reduced tissue formation over time. Interestingly, compared with levels in SW scaffolds, COL levels in the NF scaffolds remained significantly lower after 2 weeks, and this difference was even greater after 6 weeks. This finding suggests that the osteoblasts may interact with the NF PLLA matrix as they would potentially interact with a collagen substrate.

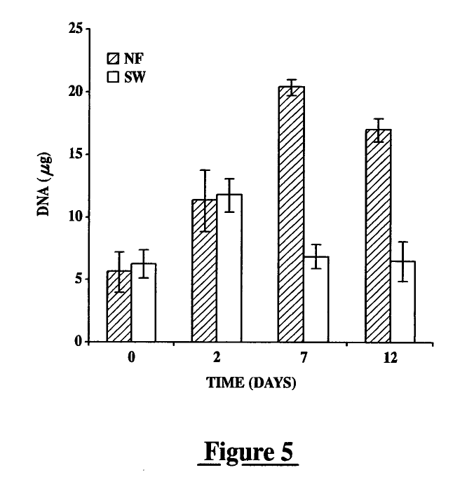

FIG. 5 shows short-term in vitro osteoblastic proliferation behavior on PLLA scaffolds after seeding with one-half million MC3T3-E1 cells.

In summary, the present disclosure has demonstrated a new technique to create three-dimensional polymer scaffolds with micrometer and/or nanometer-scaled fibers that successfully promote in vitro bone tissue regeneration. Using reverse solid freeform fabrication, scaffolds can be fabricated with intricate pore structures and designs. Osteoblast proliferation and differentiation were greatly improved in scaffolds possessing the NF morphology. Knowing that cellular response on substrates is generally dependent on the length scale of surface features, in particular, that osteoblastic tissue formation may be enhanced by nanometer-sized features, the ability to create NF features in a controlled 3-D scaffold environment is desirable for the future development of the field of bone tissue engineering.

Methods

Scaffold fabrication for cell studies. PLLA with an inherent viscosity of 1.6 was purchased from Alkermes (Cambridge, Massachusetts). Wax and polysulfonamide (PSA) for 3-D printing were purchased from Solidscape Inc. (Merrimack, N.H.). Solvents were purchased from Fisher Scientific (Pittsburgh, Pa.).

Negative molds were designed and converted into STL data using Rhinoceros (Robert McNeel & Associates, Seattle, Wash.), and then imported into Modelworks software (Solidscape) to convert the files for 3-D printing. For each layer of the mold, molten wax and PSA were printed separately in a layer-by-layer fashion using a Modelmaker II (Solidscape). PSA was dissolved in ethanol.

For NF scaffolds, a solution of PLLA in 4:1 (v/v) dioxane:methanol was stirred at 60° C. until homogeneous. Dioxane was dripped into the mold to wet the wax surface, the polymer solution was cast into the mold, and the polymer/mold composite was phase separated overnight at −20° C. The solvent was extracted with cold ethanol (−20° C.) for 1 day and ice-cold water for 1 day. Excess polymer was trimmed with a razor blade, and the polymer/mold composite was washed in 37° C. cyclohexane to dissolve the wax mold, followed by washings in 37° C. ethanol and water, and subsequent freeze-drying.

For SW scaffolds, the PLLA/dioxane solution was similarly cast and phase separated. The polymer/mold composites were lyophilized at −5 to −10° C. to remove dioxane crystallites. Excess polymer was trimmed with a razor blade and wax molds were removed similarly to those in NF samples.

Scaffold characterization. Morphology of the scaffolds was analyzed by SEM (S-3200, Hitachi, Japan) after sputter-coating with gold.

BET surface area (reproducible within about 3%) was measured by N₂ adsorption experiments at liquid nitrogen temperature on a Belsorp-Mini (Bel Japan, Osaka, Japan), after evacuating samples at 25° C. for 10 hours (<7×10⁻³ Torr).

Mold fabrication from CT scans. Images of the human ear and mandible were acquired from the National Library of Medicine's Visible Human Project. Three-dimensional reconstructions and STL conversions were performed using Mimics V8.1 (Materialise USA, Ann Arbor, Mich.) before proceeding with the SFF process.

Cell culture and differentiation. MC3T3-E1 (clone 26) cells were cultured in alpha-minimum essential media (α-MEM) supplemented with 10% fetal bovine serum (FBS), 100 U/mL penicillin and 100 μg/mL streptomycin in a humidified incubator at 37° C. with 5% CO₂.

The ethylene oxide sterilized scaffolds were soaked in a 50:50 phosphate-buffered saline (PBS):ethanol solution for about 1 hour under reduced pressure to allow the PBS:ethanol solution to penetrate the scaffold. Afterwards, the PBS:ethanol solution was exchanged with PBS 3 times for 30 minutes each on an orbital shaker (Model 3520, Lab-Line Instruments, Melrose Park, Ill.) at 70 rpm. Scaffolds were washed with complete media (α-MEM, 10% FBS, 1% antibiotics) twice for 2 hours each on an orbital shaker, transferred to custom-built Teflon trays, and seeded with 2×10⁶ MC3T3-E1 cells. After 48 hours, scaffolds were transferred into 6-well tissue culture plates containing 3 mL of complete media. After 7 days, the complete media was supplemented with 50 mg/mL ascorbic acid and 10 mM β-glycerol phosphate. Media was changed every other day.

Real-time PCR was used to detect the mRNAs encoding osteocalcin (OCN), bone sialoprotein (BSP), and type I collagen (COL) at 2 and 6 weeks. Total RNA was isolated using an RNeasy Mini Kit (Qiagen) with Rnase-Free DNase set (Qiagen, Valencia, Calif.) according to protocol after scaffolds were mechanically homogenized with a Tissue-Tearor (BioSpec Products, Inc., Bartlesville, Okla.). The cDNA was made using a Geneamp PCR (Applied Biosystems, Foster City, Calif.) with TaqMan (Applied Biosystems) reverse transcription reagents and 10 minute incubation at 25° C., 30 minute reverse transcription at 48° C., and 5 minute inactivation at 95° C. Real-time PCR was set up using TaqMan Universal PCR Master mix and specific primer sequence for OCN (5′-CCGGGAGCAGTGTGAGCTTA-3′ and 5′-TAGATGCGTTTGTAGGCGGTC-3′), BSP (5′-CAGAGGAGGCAAGCGTCACT-3′ and 5′-CTGTCTGGGTGCCAACACTG-3′), and COL (5′-GCATGGCCAAGAAGACATCC-3′ and 5′-CCTCGGGTTTCCACGTCTC-3′) with 2 minute incubation at 50° C., a 10 minute Taq Activation at 95° C., and 50 cycles of denaturation for 15 seconds at 95° C. followed by an extension for 1 minute at 72° C. on an ABI Prism 7500 Real-Time PCR System (Applied Biosystems). Target genes were normalized against GAPDH (Applied Biosystems).

After 6 weeks of culture, scaffolds for mineralization quantification were washed 3 times for 5 minutes each in double-distilled water and then homogenized with a Tissue-Tearor in 1 mL of double-distilled water. Samples were then incubated in 0.5 M acetic acid overnight. Total calcium content of each scaffold was determined by o-cresolphthalein-complexone method following the manufacturer's instructions (Calcium LiquiColor, Stanbio Laboratory, Boerne, Tex.).

Histological samples were fixed in 10% neutral buffered formalin solution (Sigma, St. Louis, Mo.), dried through an ethanol gradient, and embedded in paraffin. Embedded samples were cut into 5 μm sections and stained with Hematoxylin and Eosin-Phloxine or 5% silver nitrate and nuclear fast red solution. All samples were run at n=3, and experiments were performed twice to ensure reproducibility.

While several embodiments have been described in detail, it will be apparent to those skilled in the art that the disclosed embodiments may be modified. Therefore, the foregoing description is to be considered exemplary rather than limiting.

What is claimed is:

1. A method for forming a porous three-dimensional (3-D) object, comprising:
creating a mold from a negative replica of the 3-D object, the 3-D object having a first size and at least one predetermined feature;
casting a flowable material at least one of into or onto the mold; and
forming pores of at least one of a second size or a third size in the flowable material, thereby forming the porous 3-D object.

2. The method as defined in claim 1, further comprising removing the mold from the porous 3-D object.

3. The method as defined in claim 2 wherein removing the mold is accomplished by at least one of dissolution, melting, sublimation, evaporation, burning, or combinations thereof.

4. The method as defined in claim 1, wherein before creating the mold, the method further comprises at least one of designing or obtaining a 3-D image of the 3-D object.

5. The method as defined in claim 1 wherein at least one of the first size, the second size or the third size is different from at least an other of the first size, the second size or the third size.

6. The method as defined in claim 1 wherein the first size is greater than the second size, and the second size is greater than the third size.

7. The method as defined in claim 1 wherein the 3-D object has at least one of predetermined mechanical properties, predetermined physical properties, predetermined physiological properties, predetermined biological properties, predetermined chemical properties, or combinations thereof.

8. The method as defined in claim 1 wherein forming the pores further includes forming pre-designed interconnected open pores.

9. The method as defined in claim 1 wherein forming the pores is accomplished by at least one of phase separation, evaporation, sublimation, etching, gas generation, particulate-leaching, or combinations thereof.

10. The method as defined in claim 1, further comprising:
introducing a porogen material to the mold prior to casting the flowable material; and
removing the porogen material from the 3-D object.

11. The method as defined in claim 1 wherein the pores are formed in the flowable material prior to casting the flowable material.

12. The method as defined in claim 1 wherein the pores are formed in the flowable material after casting the flowable material.

13. The method as defined in claim 1 wherein the flowable material is selected from synthetic polymers, natural macromolecules, natural polymers, organic compounds, inorganic compounds, metals, and combinations thereof.

14. A porous 3-D object formed by the process as defined in claim 1.

15. A porous three-dimensional (3-D) object, comprising:
a solidified flowable material having a first size and at least one predetermined feature defined by a mold having a negative replica of the three-dimensional object; and
a plurality of pores defined throughout the solidified flowable material, at least some of the plurality of pores having a second size, and at least some other of the plurality of pores having a third size.

16. The porous 3-D object as defined in claim 15 wherein the first size is greater than or equal to about 10⁻³, wherein the second size ranges from about 10⁻⁶ m to about 10⁻³ m, and wherein the third size ranges from about 10⁻⁹ m to about 10⁻⁶ m.

17. The porous 3-D object as defined in claim 15 wherein the 3-D object has at least one of predetermined mechanical properties, predetermined physical properties, predetermined physiological properties, predetermined biological properties, predetermined chemical properties, or combinations thereof.

18. The porous 3-D object as defined in claim 15, further comprising at least one of the plurality of pores having a shape selected from a computer designed shape, a porogen-defined shape, a predesigned interconnected open pore shape, and combinations thereof.

19. The porous 3-D object as defined in claim 15 wherein the flowable material is selected from synthetic polymers, natural macromolecules, natural polymers, organic compounds, inorganic compounds, metals, and combinations thereof.

20. The porous 3-D object as defined in claim 15 wherein the porous 3-D object is a three-dimensional polymer scaffold with micrometer and nanometer-scaled fibers.

21. A method of forming a 3-D object, comprising:
designing or obtaining a 3-D image of the 3-D object, the 3-D object having a first size and at least one predetermined feature;
creating a mold from a negative replica of the 3-D image;
casting a flowable material at least one of into or onto the mold;
forming pores of at least one of a second size or a third size in the flowable material, thereby forming the porous 3-D object; and
removing the mold from the porous 3-D object.

22. The method as defined in claim 21 wherein removing the mold is accomplished by at least one of dissolution, melting, sublimation, evaporation, burning, or combinations thereof.

23. The method as defined in claim 21 wherein at least one of the first size, the second size or the third size is different from at least an other of the first size, the second size or the third size.

24. The method as defined in claim 23 wherein the first size is greater than the second size, and the second size is greater than the third size.

25. The method as defined in claim 21 wherein forming the pores further includes forming pre-designed interconnected open pores.

26. The method as defined in claim 21 wherein forming the pores is accomplished by at least one of phase separation, evaporation, sublimation, etching, gas generation, particulate-leaching, or combinations thereof.

27. The method as defined in claim 21, further comprising:
introducing a porogen material to the mold prior to casting the flowable material; and
removing the porogen material from the 3-D object.

28. The method as defined in claim 21 wherein the pores are formed in the flowable material prior to casting the flowable material or after casting the flowable material.

29. The method as defined in claim 21 wherein the flowable material is selected from synthetic polymers, natural macromolecules, natural polymers, organic compounds, inorganic compounds, metals, and combinations thereof.

30. A porous 3-D object formed by the process as defined in claim 21.


SEQUENCE LISTING (6 sequences)

SEQ ID NO: 1 - DNA, 20 bp, Artificial Sequence
  Specific primer sequence for osteocalcin
ccgggagcagtgtgagctta

SEQ ID NO: 2 - DNA, 21 bp, Artificial Sequence
  Specific primer sequence for osteocalcin
tagatgcgtttgtaggcggtc

SEQ ID NO: 3 - DNA, 20 bp, Artificial Sequence
  Specific primer sequence for bone sialoprotein
cagaggaggcaagcgtcact

SEQ ID NO: 4 - DNA, 20 bp, Artificial Sequence
  Specific primer sequence for bone sialoprotein
ctgtctgggtgccaacactg

SEQ ID NO: 5 - DNA, 20 bp, Artificial Sequence
  Specific primer sequence for collagen
gcatggccaagaagacatcc

SEQ ID NO: 6 - DNA, 19 bp, Artificial Sequence
  Specific primer sequence for collagen
cctcgggtttccacgtctc